(19) United States
(12) Patent Application Publication
(10) Pub. No.: US 2016/0237407 A1
(43) Pub. Date: Aug. 18, 2016

(54) UNIVERSAL DONOR CHIMERIC ANTIGEN RECEPTOR CELLS

(71) Applicant: Batu Biologics, Inc., San Diego, CA (US)

(72) Inventors: Samuel C. Wagner, San Diego, CA (US); Thomas E. Ichim, San Diego, CA (US); Santosh Kesari, San Diego, CA (US); Boris Minev, San Diego, CA (US); Julia S. Szymanski, San Diego, CA (US)

(73) Assignee: Batu Biologics, Inc., San Diego, CA (US)

(21) Appl. No.: 15/046,259

(22) Filed: Feb. 17, 2016

Related U.S. Application Data
(60) Provisional application No. 62/117,161, filed on Feb. 17, 2015.

(51) Int. Cl. C12N 5/0783; C07K 16/30; C07K 14/725; C07K 16/32

(52) U.S. Cl. CPC C12N 5/0636; C07K 16/32; C07K 16/30; C07K 14/7051; C12N 2510/00; C12N 2501/48; C12N 2501/515; C12N 2501/51; C12N 2501/2307; C12N 2501/2302; C12N 2506/11; C12N 2506/1369; C07K 2319/03; C07K 2319/715

(57) ABSTRACT

Disclosed are allogeneic cells useful for the treatment of cancer in a universal donor, off the shelf, manner. In one embodiment of the invention cord blood derived T cell progenitors are matured with anti-CD3 and anti-CD28, interleukin-7 and transfected with a construct encoding a chimeric antigen receptor (CAR) targeting a tumor antigen or a tumor endothelial associated antigen on the antigen binding domain. The intracellular domain containing CD3 zeta chain and at least one shRNA domain encoding a transcript which generates at least one siRNA capable of inhibiting expression of HLA I and/or HLA II. In another embodiment mesenchymal stem cells are transfected with CAR to enhance migration into tumors and induce tumor death, reduction of inflammation, or immune sensitization. In another embodiment universal donor CAR-MSC are disclosed.

### CROSS-REFERENCE TO RELATED APPLICATION

This application claims the benefit of U.S. Provisional Application No. 62/117,161 filed on Feb. 17, 2015, the contents of which are incorporated herein by reference in its entirety.

### FIELD OF THE INVENTION

The invention pertains to the field of anti-tumor immunity, more specifically, the invention pertains to the field of induction of antitumor immunity utilizing CAR-MSC to become activated subsequent to contact with a tumor, said activation inducing Type 1 immunity and tumor inhibition. The invention additionally pertains to the field of CAR-T cells derived from umbilical cord blood and lacking immunogenicity.

### BACKGROUND OF THE INVENTION

Utilization of CAR-T cells has led to a revolution in cancer therapeutics. The original concept of antitumor immunity began in part by observations of tumor infiltrating lymphocytes, that is, in a wide variety of tumors, lymphocytic infiltration was observed and associated with positive prognosis. This has been observed in bowel tumors, head and neck cancer, bladder cancer, glioblastoma, breast cancer, melanoma, lung cancer, stomach cancer, ovarian cancer, and colorectal cancer.

In some situations it has been demonstrated that immune stimulation through vaccination or immunotherapy results in augmentation of tumor infiltrating lymphocytes. Furthermore, in patients who undergo spontaneous regressions of cancer, said regressions are associated with lymphocytic infiltrates. Furthermore, in some patients spontaneous regression occurs after bacterial or viral infections, further suggesting immunological causes. In addition to lymphocytic infiltrations, antigen-specific T cells have been detected to be associated with spontaneous regression. Studies initiated by Rosenberg's group demonstrated that extraction of tumor infiltrating lymphocytes followed by ex vivo expansion and re-infusion results in substantial tumor regression, especially when patients are previously treated by lymphodepletion.

Augmentation of activity of lymphocyte immunotherapies was observed utilizing chimeric receptors was demonstrated in animal studies, Hwu et al examined the in vivo activity of murine T cells transduced with a chimeric receptor gene (MOv-gamma) derived from the mAb MOv18, which binds to a folate-binding protein overexpressed on most human ovarian adenocarcinomas. Nude mice that were given i.p. implants of human ovarian cancer (IGROV) cells were treated 3 days later with i.p. murine tumor-infiltrating lymphocytes (TIL) derived from an unrelated tumor. Mice treated with MOv-gamma-transduced TIL (MOv-TIL) had significantly increased survival compared to mice treated with saline only, nontransduced TIL, or TIL transduced with a control anti-trinitrophenyl chimeric receptor gene (TNP-TIL). In another model, C57BL/6 mice were given i.v. injections of a syngeneic methylcholanthrene-induced sarcoma transduced with the folate-binding protein (FBP) gene. Three days later, mice were treated i.v. with various transduced murine TIL (derived from an unrelated tumor), followed by low-dose systemic interleukin 2. Eleven days after tumor injection, mice were sacrificed, and lung metastases were counted. In multiple experiments, mice receiving MOv-TIL had significantly fewer lung metastases than did mice treated with interleukin 2 alone, nontransduced TIL, or TNP-TIL. These studies indicate that T cells can be gene modified to react in vivo against tumor antigens, defined by mAbs. This approach is potentially applicable to a number of neoplastic and infectious diseases and may allow adoptive immunotherapy against types of cancer not previously amenable to cellular immunotherapy.

Researchers have attempted to counter the immune system's tolerance to cancer cell antigens by genetically modifying T cells with a chimeric antigen receptor (CAR) via grafting, called CAR-T cells. CAR-T have the advantage of not requiring presentation of tumor antigen on MHC since they possess an antibody domain. CAR are usually generated by joining a single chain antibody (scFv) to an intracellular signaling domain, usually the zeta chain of the TCR/CD3 complex. The most recent construction of CARs also contain a co-stimulatory molecule such as CD28 or 41BB that can improve effector cell survival and proliferation. For cancer therapy, T-CARs have at least three major advantages over natural T cell receptors.

First, the antigen binding affinity of scFv is typically much higher than the binding moiety of most TCRs. A high affinity binding is desired for efficient T cell activation. Second, due to the nature of scFv-mediated antigen binding, T-CAR recognition is non-MHC restricted and independent of antigen processing. This widens the use of T-CARs to patients with different MHC haplotypes. Third, because T-CAR recognition is non-MHC restricted, their ability to target cancer cells is not hampered by a cancer cells' ability to down regulate MHC (an important mechanism by which tumor cells evade cancer immunotherapies). CARs have been previously constructed with scFvs that bind to a variety of tumor-associated antigens. Encouraging preclinical data has prompted a series of clinical trials using adoptive transfer of T cells engrafted with these CARs for treatment of tumors having different tissue origins, including melanoma, lymphoma, neuroblastoma, and colorectal cancer. Many of these trials have shown promising results, even complete remission of the established tumors in some cases.

Despite the impressive improvement of T-CARs over native T effector cells, there are significant drawbacks. For example, T-CARs do not actively migrate to the tumor site and they lack an active mechanism to extravasate into tumor tissue. Unfortunately current CAR-T cell approaches are limited by lack of ability to generate “universal donor” CAR-T cells, as well as by general toxicity in some cases or lack of efficacy in others.

### DETAILED DESCRIPTION OF THE INVENTION

Included in the scope of the invention are functional portions of the inventive CARs described herein. In one embodiment said CAR is utilized to activate T cells to endow cytokine production or to stimulate cytotoxicity against tumors. In another embodiment CAR is utilized to activate mesenchymal stem cells (MSC) to preferentially migrate to tumors.

In another embodiment, CAR transfected MSC are generated with the antigen binding domain of CAR binding to a tumor antigen and the signaling domain activating MSC to produce type 1 cytokines. Numerous intracellular domains may be generated including activation of STAT 6 through the JAK-STAT pathway. The definition “functional portion” when used in reference to a CAR refers to any part or fragment of the CAR of the invention, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, 25%, 30%, 50%, 68%, 80%, 90%, 95%, or more, of the parent CAR.

The functional portion can comprise additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer. In one embodiment of the invention the ability of MSC to inhibit cancer cell growth is amplified by transfection with CAR, wherein CAR intracellular domain activates tumoricidal genes such as TRAIL, TNF, Type 1 or Type 2 interferons. Basal cancer inhibitory properties of MSC are described in the art and may be incorporated for the practice of the invention. Means of transfecting MSC with tumoricidal, tumor inhibitory, or immune stimulatory genes are described by others in the art and applicable to the practice of the current invention. Utilization of TRAIL, IL-12, IL-21, suicide gene, IL-18, TNF-alpha, interferon beta, single chain antibodies, and endostatin as tumor targeting agents delivered by MSC has been previously described and incorporated by reference.

More desirably, the additional amino acids enhance the biological activity, as compared to the biological activity of the parent CAR. Within the scope of the present disclosure are functional variants of the inventive CARs described herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, 50%, 75%, 80%, 90%, 98% or more identical in amino acid sequence to the parent CAR. A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the inventive CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The CAR can consist essentially of the specified amino acid sequence or sequences described herein, such that other components, e.g., other amino acids, do not materially change the biological activity of the functional variant. The CARs of the invention retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CARs of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, .alpha.-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, beta-phenylserine beta-hydroxyphenylalanine, phenylglycine, alpha-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, alpha-aminocyclopentane carboxylic acid, alpha-aminocyclohexane carboxylic acid, alpha-aminocycloheptane carboxylic acid, alpha-(2-amino-2-norbornane)-carboxylic acid, alpha, gamma-diaminobutyric acid, alpha, beta-diaminopropionic acid, homophenylalanine, and alpha-tert-butylglycine.

The CARs of embodiments of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CARs of embodiments of the invention (including functional portions and functional variants thereof) can be obtained by methods known in the art. The CARs may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Further, some of the CARs of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies, such as Synpep (Dublin, Calif.), Peptide Technologies Corp. (Gaithersburg, Md.), and Multiple Peptide Systems (San Diego, Calif.). In this respect, the inventive CARs can be synthetic, recombinant, isolated, and/or purified.

An embodiment of the invention further provides an antibody, or antigen binding portion thereof, which specifically binds to an epitope of the CARs of the invention. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for the functional portion of the inventive CAR.

In an aspect, the disclosure provides methods for inducing ex vivo expansion of a population of T cells. T cell proliferation can be induced without the need for antigen, thus providing an expanded T cell population which is polyclonal with respect to antigen reactivity. Embodiments of the method provide for sustained proliferation of a population of T cells over an extended period of time to yield a multi-fold increase in the number of cells relative to the original T cell population. This aspect can comprise a method of enhancing ex vivo proliferation of a T cell population comprising contacting the T cell population with IL-7, an anti-CD3 antibody, and an anti-CD28 antibody, or functional fragments thereof, to activate and expand the T cell population. In some embodiments, the T cell population may be further contacted with IL-2. Suitably an increased population of CD3/CD28-expanded T cells is generated by the method. In certain embodiments, methods are provided for generating a population of CD3/CD28-expanded T cells, the methods comprising contacting the T cell population with IL-7 and anti-CD3/CD28 antibodies to activate and expand the T cell population. Embodiments provide for a T cell population that is taken or isolated from cord blood. In some embodiments the method provides for the generation of activated and expanded T cells in about 10 to about 20 days, and in some embodiments about 14 days.

As used herein, a “CD3/CD28-expanded T cell” refers to a T cell that has been co-stimulated by contact with anti-CD3 and anti-CD28 antibodies. As used herein anti-CD3, anti-CD28, and anti-CD3/CD28 antibodies refer to any molecule or complex that interacts with both CD3 and CD28 receptors on the T cell. While it is shown below in the Examples that T cells may be contacted with beads with anti-CD3/CD28 antibodies, it is envisioned that the antibodies may be presented on surfaces including but not limited to particles, beads, and cells. Hence, T cells may be contacted with any molecule or complex that interacts with both CD3 and CD28 receptors on the T cell, wherein the complexes may be presented on surfaces including but not limited to particles, beads, and cells.

In some embodiments, a population of T cells is induced to proliferate (or “expand,” “propagate,” “grow,” and the like) by contacting the T cells with IL-7 in combination with a molecule that can active the T cells and with a molecule that can stimulate the T cells under conditions suitable for inducing expansion of at least one T cell, or a portion, a plurality, a majority, or substantially all T cells that contact the molecule that can activate and the molecule that can stimulate the T cell(s). The contacting of the T cell can be accomplished by any suitable method known in the art, either sequentially or simultaneously. In embodiments that comprise sequential contacting strategies, the T cell is suitably first contacted with an agent that can activate the T cell and subsequently contacted with an agent that can stimulate the T cell and induce proliferation.

In some embodiments, activation of a population of T cells is accomplished by contacting the T cells with a first agent which induces or activates a TCR/CD3 complex-associated signal in the T cells. In embodiments the activation of the TCR/CD3 complex-associated signal in a T cell can be accomplished either by ligation of the T cell receptor (TCR)/CD3 complex, or by directly stimulating receptor-coupled signaling pathways. In embodiments, an anti-CD3 antibody can be used to activate a population of T cells.

In some embodiments proliferation of an activated T cell population can be induced to proliferate by contacting the activated T cells with a second agent which stimulates an accessory molecule on the surface of the T cells. In embodiments a population of CD4+ T cells can be stimulated to proliferate with an anti-CD28 antibody directed to the CD28 molecule on the surface of the T cells. Embodiments also provide for stimulation by other natural ligands for CD28, which can be soluble, on a cell membrane, or coupled to a solid phase surface. In some embodiments, proliferation of an activated population of T cells can be induced by stimulation of one or more intracellular signals which result from ligation of an accessory molecule.

In some embodiments, the agent provides the primary activation signal and the agent providing the co-stimulatory agent can be added either in soluble form or coupled to a solid phase surface. In some embodiments, the two agents are coupled to the same solid phase surface such as, for example, the surface of a cell culture vessel or a particle (e.g., microparticle, nanoparticle, beads including magnetic beads, polymeric beads, glass beads, and the like). In embodiments the methods can comprise contacting a costimulatory signal to a T cell for T cell expansion (e.g., an anti-CD28 antibody or an active fragment thereof), coupled to a solid phase surface which may additionally include an agent that provides a primary activation signal to the T cell (e.g., an anti-CD3 antibody or an active fragment thereof) coupled to the same solid phase surface. In some embodiments the agents are attached to beads. Compositions comprising each agent coupled to different solid phase surfaces (i.e., an agent that provides a primary T cell activation signal coupled to a first solid phase surface and an agent that provides a costimulatory signal coupled to a second solid phase surface) are also within the scope of the disclosure.

Following activation and stimulation of the T cells, the proliferation of the T cells in response to continuing exposure to the agents can be monitored by any suitable method known in the art. When the rate of T cell proliferation decreases, the T cells can be reactivated and restimulated, such as with additional anti-CD3 antibody and anti-CD28 antibody, or active fragments thereof, to induce further proliferation. In an embodiment, the rate of T cell proliferation is monitored by examining cell size. Alternatively, an embodiment provides for monitoring T cell proliferation by assaying for expression of cell surface molecules in response to exposure to the molecules, such as anti-CD3/CD28 antibodies. The monitoring and restimulation of the T cells can be repeated for sustained proliferation to produce a population of T cells increased in number from about 100- to about 100,000-fold or more relative to the original T cell population. As noted above, some embodiments provide for methods that expand the T cell population in about 7 days, about 10 days, about 14 days, or about 20 days.

The method of the invention can be used to expand selected T cell populations for use in treating a disease or disorder such as, for example lymphopenia or cancer. In embodiments that relate to the treatment of a disease, the method suitably comprises priming one or a plurality of the expanded T cell population with an antigen of interest such as, for example, a cancer cell, under conditions that produce an antigen-specific T cell population. The resulting T cell population can be used for therapy or can be used for in vitro analysis of the disease, such as cancer. In embodiments, a population of tumor-infiltrating lymphocytes can be obtained from a subject afflicted with cancer and the T cells stimulated to proliferate to sufficient numbers and restored to the subject.

The term “T cell activation” is used herein to define a state in which a T cell response has been initiated or activated by a primary signal, such as through the TCR/CD3 complex, but not necessarily due to interaction with a protein antigen. A T cell is activated if it has received a primary signaling event which initiates an immune response by the T cell. In embodiments, T cell activation can be accomplished by stimulating the T cell TCR/CD3 complex. An anti-CD3 monoclonal antibody can be used to activate a population of T cells via the TCR/CD3 complex. A number of anti-human CD3 monoclonal antibodies are commercially available. Other antibodies which bind to the same epitopes as an anti-CD3 antibody can also be used. Additional antibodies, or combinations of antibodies, can be prepared and identified by techniques known in the art.

The activated population of T cells can be induced to proliferate (i.e., a population of T cells that has received a primary activation signal induced by an anti-CD3 antibody) by stimulation of the accessory molecule CD28 by contacting an activated population of T cells with a ligand which binds CD28. In embodiments, an anti-CD28 monoclonal antibody or fragment thereof capable of crosslinking the CD28 molecule, or a natural ligand for CD28 (e.g., a member of the B7 family of proteins, such as B7-1(CD80) and B7-2 (CD86) (Freedman, A. S. et al. (1987) J. Immunol. 137:3260-3267; Freeman, G. J. et al. (1989) J. Immunol. 143:2714-2722; Freeman, G. J. et al. (1991) J. Exp. Med. 174:625-63 1; Freeman, G. J. et al. (1993) Science 262:909-911; Azuma, M. et al. (1993) Nature 366:76-79; Freeman, G. J. et al. (1993) J. Exp. Med. 178:2185-2192)) can be used to induce stimulation of the CD28 molecule. In embodiments the molecule comprises an anti-CD28 antibody or an active fragment thereof. A number of anti-CD28 antibodies are known in the art and are commercially available.

For T cell costimulation, IL-7 and the agents that activate and induce expansion can be provided to the T-cells, and incubated with the T cells to be co-stimulated. The ratio of T cells to stimulating agents can vary widely, depending on the source of the agent(s). In embodiments comprising use of soluble agents (e.g., anti-CD3/CD28 antibodies), soluble agents are added to the T cell culture in an amount sufficient to result in co-stimulation of activated T cells, in combination with IL-7. The appropriate amount of soluble agent to be added will vary with the specific agent, but can be determined by assaying different amounts of the soluble agent in T cell cultures and measuring the extent of co-stimulation by proliferation assays or production of cytokines. Typically in embodiments comprising anti-CD3 and anti-CD28 antibodies, such agents can be provided at concentrations typically ranging from 0.01 ng to about 100 mg/nL, or to about 100 ng/mL, or in some embodiments from about 10 ng to about 0 50 mg/mL. In embodiments comprising one or more agents attached to a substrate such as, for example ClinExVivo Dynabeads (Dynal/Invitrogen Corp.), an excess number of beads per cell in culture can be provided, such as about 2:1, 3:1, 4:1, 5:1, 6:1, 7:1, 8:1, 9:1, 10:1, 15:1, 20:1, 25:1, or even 50:1, or more beads:T cell (as measured in the initial culture). In some embodiments, the amount of the activation and/or stimulatory agents can be determined and/or adjusted based on the response of the culture after contacting, as measured by the T cell response. Similarly the amount of IL-7 contacted with the T cell population can vary from about 0.01 ng to about 100 mg/mL, or in some embodiments from about 1.0 ng to about 100 ng/mL, or about 1.0 ng to about 10.0 ng/mL.

In another embodiment, a natural ligand of CD28 (B7-1, B7-2) can be presented to T cells in a form attached to a solid phase surface, such as beads. These molecules can then be attached to the solid phase surface via conventional techniques (e.g., covalent modification using tosyl linkage to tosyl activated magnetic immunobeads (Dynal Inc., Great Neck, N.Y.) according to manufacturer's instructions.) The molecules may also be immobilized on modified polystyrene beads or culture vessel surfaces (e.g., through an avidin- or streptavidin-biotin complex). In such embodiments, the soluble molecule(s) can be crosslinked to biotin and then reacted with the solid phase surface to which avidin or streptavidin molecules are bound. Conversely, the soluble molecules can be crosslinked to avidin or streptavidin and reacted with a solid phase surface that is derivatized with biotin molecules.

In one embodiment of the invention mesenchymal stem cells are transfected with a CAR capable of endowing said MSC with ability to trigger a T cell mediated immune response. In one embodiment the CAR acts as a means of attaching MSC to cancer cells. In another embodiment, the CAR acts as a means of triggering enhanced adhesion of said MSC to cancer cells. In one specific embodiment CAR consists of an extracellular domain capable of binding tumor antigen, specifically HER2, and an intracellular domain comprising of the intracellular domain of TLR-4. There are several methods known in the art for the generation of MSC. In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2 ' 107 cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1 ' 106 cells per ml in 175 cm2 tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1 ' 106 per 175 cm2. BM-MSC are subsequently transfected with CAR gene. In some embodiments of the invention transfection is accomplished by use of lentiviral vectors, said means to perform lentiviral mediated transfection are well-known in the art and discussed in the following references. Some specific examples of lentiviral based transfection of genes into MSC include transfection of SDF-1 to promote stem cell homing, particularly hematopoietic stem cells, FGF-18 to promote osteogenic differentiation, GDNF to treat Parkinson's in an animal model, HGF to accelerate remyelination in a brain injury model, akt to protect against pathological cardiac remodeling and cardiomyocyte death, TRAIL to induce apoptosis of tumor cells, PGE-1 synthase for cardioprotection, NUR77 to enhance migration, BDNF to reduce ocular nerve damage in response to hypertension, HIF-1 alpha to stimulate osteogenesis dominant negative CCL2 to reduce lung fibrosis, interferon beta to reduce tumor progression, HLA-G to enhance immune suppressive activity, hTERT to induce differentiation along the hepatocyte lineage, cytosine deaminase, OCT-4 to reduce senescence, BAMBI to reduce TGF expression and protumor effects, HO-1 for cardioprotection, LIGHT to induce antitumor activity, miR-126 to enhance angiogenesis, bcl-2 to induce generation of nucleus pulposus cells, telomerase and myocardin to induce cardiogenesis, CXCR4 to accelerate hematopoietic recovery and reduce renal allograft rejection, wnt11 to promote chondrogenesis, Islet-1 to promote pancreatic differentiation, IL-27 to reduce autoimmune disease, ACE-2 to reduce sepsis, CXCR4 to reduce liver failure, and lung injury, and the HGF antagonist NK4 to reduce cancer.

Cell cultures are tested for sterility weekly, endotoxin by limulus amebocyte lysate test, and mycoplasma by DNA-fluorochrome stain.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm2 flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington DC, USA). MSCs harvested from each 10-flask set were stored for up to 4 h at 4° C. and combined at the end of the harvest. A total of 2-10 ' 106 MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment of the current invention radiotherapy is utilized to direct MSC expressing CAR to tumors. It was previously reported using MSC labeled with a lipophilic dye that irradiation increases migration efficacy into colon cancer xenografts. MSC were shown to migrate to tumor xenografts (LoVo) of various origins, with few cells found in normal tissues. A lentiviral vector efficiently transduced MSCs in the presence, but not the absence, of hexadimethrine bromide (Polybrene). When LoVo cells were treated with increasing radiation doses, more MSC were found to migrate to them than to untreated tumors. Irradiation increased MSC localization in HT-29 and MDA-MB-231, but not UMSCC1, xenografts. Monocyte chemotactic protein-1 was modestly elevated in irradiated tumors. Media from irradiated LoVo cells stimulated MSC invasion into basement membranes. Clinical and clinically relevant means of directing MSC into tumors has been previously described.

In one embodiment of the invention CAR-MSC are transfected with anti-apoptotic proteins to enhance in vivo longevity. The present invention includes a method of using CAR-MSC that have been cultured under conditions to express increased amounts of at least one anti-apoptotic protein as a therapy to inhibit or prevent apoptosis. In one embodiment, the CAR-MSC which are used as a therapy to inhibit or prevent apoptosis have been contacted with an apoptotic cell. The invention is based on the discovery that CAR-MSC that have been contacted with an apoptotic cell express high levels of anti-apoptotic molecules. In some instances, the CAR-MSC that have been contacted with an apoptotic cell secrete high levels of at least one anti-apoptotic protein, including but not limited to, STC-1, BCL-2, XIAP, Survivin, and Bcl-2XL. Methods of transfecting antiapoptotic genes into MSC have been previously described which can be applied to the current invention, said antiapoptotic genes that can be utilized for practice of the invention, in a nonlimiting way, include GATA-4, FGF-2, bcl-2, and HO-1. Based upon the disclosure provided herein, CAR-MSC can be obtained from any source. The CAR-MSC may be autologous with respect to the recipient (obtained from the same host) or allogeneic with respect to the recipient. In addition, the CAR-MSC may be xenogeneic to the recipient (obtained from an animal of a different species). In one embodiment of the invention CAR-MSC are pretreated with agents to induce expression of antiapoptotic genes, one example is pretreatment with exendin-4 as previously described. In a further non-limiting embodiment, CAR-MSC used in the present invention can be isolated, from the bone marrow of any species of mammal, including but not limited to, human, mouse, rat, ape, gibbon, bovine. In a non-limiting embodiment, the CAR-MSC are isolated from a human, a mouse, or a rat. In another non-limiting embodiment, the CAR-MSC are isolated from a human.

Based upon the present disclosure, CAR-MSC can be isolated and expanded in culture in vitro to obtain sufficient numbers of cells for use in the methods described herein provided that the CAR-MSC are cultured in a manner that promotes contact with a tumor endothelial cell. For example, CAR-MSC can be isolated from human bone marrow and cultured in complete medium (DMEM low glucose containing 4 mM L-glutamine, 10% FBS, and 1% penicillin/streptomycin) in hanging drops or on non-adherent dishes. The invention, however, should in no way be construed to be limited to any one method of isolating and/or to any culturing medium. Rather, any method of isolating and any culturing medium should be construed to be included in the present invention provided that the CAR-MSC are cultured in a manner that provides CAR-MSC to express increased amounts of at least one anti-apoptotic protein. Culture conditions for growth of clinical grade MSC have been described in the literature and are incorporated by reference.

Any medium capable of supporting CAR-MSC in vitro may be used to culture the CAR-MSC. Media formulations that can support the growth of CAR-MSC include, but are not limited to, Dulbecco's Modified Eagle's Medium (DMEM), alpha modified Minimal Essential Medium (alpha MEM), and Roswell Park Memorial Institute Media 1640 (RPMI Media 1640) and the like. Said media and conditions for culture of MSC-and by virtue of the invention CAR-MSC are known in the art. Typically, up to 20% fetal bovine serum (FBS) or 1-20% horse serum is added to the above medium in order to support the growth of CAR-MSC. A defined medium, however, also can be used if the growth factors, cytokines, and hormones necessary for culturing CAR-MSC are provided at appropriate concentrations in the medium. Media useful in the methods of the invention may contain one or more compounds of interest, including, but not limited to, antibiotics, mitogenic or differentiation compounds useful for the culturing of CAR-MSC. The cells may be grown at temperatures between 27° C. to 40° C., preferably 31° C. to 37° C., and more preferably in a humidified incubator. The carbon dioxide content may be maintained between 2% to 10% and the oxygen content may be maintained between 1% and 22%. The invention, however, should in no way be construed to be limited to any one method of isolating and culturing CAR-MSC. Rather, any method of isolating and culturing CAR-MSC should be construed to be included in the present invention.

Antibiotics which can be added into the medium include, but are not limited to, penicillin and streptomycin. The concentration of penicillin in the culture medium, in a non-limiting embodiment, is about 10 to about 200 units per ml. The concentration of streptomycin in the culture medium is, in a non-limiting embodiment, about 10 to about 200 mu g/ml.

CAR-MSC which express increased amounts of at least one anti-apoptotic protein may be administered to an animal in an amount effective to provide a therapeutic effect. The animal may be a mammal, including but not limited to, human and non-human primates.

The CAR-MSC can be suspended in an appropriate diluent. Suitable excipients for injection solutions are those that are biologically and physiologically compatible with the CAR-MSC and with the recipient, such as buffered saline solution or other suitable excipients. The composition for administration can be formulated, produced, and stored according to standard methods complying with proper sterility and stability. The CAR-MSC may have one or more genes modified or be treated such that the modification has the ability to cause the CAR-MSC to self-destruct or “commit suicide” because of such modification, or upon presentation of a second drug (eg., a prodrug) or signaling compound to initiate such destruction of the CAR-MSC.

The dosage of the CAR-MSC varies within wide limits and may be adjusted to the individual requirements in each particular case. The number of cells used depends on the age, weight, sex, and condition of the recipient, the number and/or frequency of administrations, the disease or disorder being treated, and the extent or severity thereof, and other variables known to those of skill in the art.

In a non-limiting embodiment, the CAR-MSC may be administered in combination with other drugs which possess anti-cancer activity. Said drugs include alkylating agents such as ifosfamide, nimustine hydrochloride, cyclophosphamide, dacarbazine, melphalan, and ranimustine, antimetabolites such as gemcitabine hydrochloride, enocitabine, cytarabine ocfosfate, a cytarabine formulation, tegafur/uracil, a tegafur/gimeracil/oteracil potassium mixture, doxifluridine, hydroxycarbamide, fluorouracil, methotrexate, and mercaptopurine, antitumor antibiotics such as idarubicin hydrochloride, epirubicin hydrochloride, daunorubicin hydrochloride, daunorubicin citrate, doxorubicin hydrochloride, pirarubicin hydrochloride, bleomycin hydrochloride, peplomycin sulfate, mitoxantrone hydrochloride, and mitomycin C, alkaloids such as etoposide, irinotecan hydrochloride, vinorelbine tartrate, docetaxel hydrate, paclitaxel, vincristine sulfate, vindesine sulfate, and vinblastine sulfate, hormone therapy agents such as anastrozole, tamoxifen citrate, toremifene citrate, bicalutamide, flutamide, and estramustine phosphate, platinum complexes such as carboplatin, cisplatin, and nedaplatin, angiogenesis inhibitors such as thalidomide, neovastat, and bevacizumab, L-asparaginase etc., drugs inhibiting the activity or production of the above bioactive substances, such as, for example, antibodies and antibody fragments that neutralize the above bioactive substances, and substances that suppress expression of the above bioactive substances, such as an siRNA, a ribozyme, an antisense nucleic acid (including RNA, DNA, PNA, and a composite thereof), substances that have a dominant negative effect such as a dominant negative mutant, vectors expressing same, cell activity inhibitors such as a sodium channel inhibitor, cell-growth inhibitors, and apoptosis inducers such as compound 861 and gliotoxin.

1. A cord blood derived T cell possessing reduced immunogenicity as compared to peripheral blood T cells, said cord blood derived T cell expressing an antigen binding domain, a transmembrane domain, and an intracellular signaling domain.

2. The cord blood derived T cell of claim 1, wherein said T cell is isolated from cord blood through selection for a T cell associated molecule.

3. The cord blood derived T cell of claim 1, wherein said T cell associated molecule is selected from a group comprising of: CD2, CD3, CD4, CD8, CD7, CD16, CD44, CD62 ligand, CD97, CD117, CD123, CD127, CXCR4, NKG2D, and the T cell receptor alpha, T cell receptor beta, or T cell receptor alpha/beta chain.

4. The cord blood derived T cell of claim 1, wherein a costimulatory molecule is inserted into said CAR.

5. The cord blood derived T cell of claim 4, wherein said costimulatory molecule is selected from a group of molecules comprising of: CD27, CD28, 4-1BB, OX40, CD30, CD40, PD-1, ICOS, CD80, CD86, lymphocyte function-associated antigen-1 (LFA-1), CD2, CD7, LIGHT, NKG2C, B7-H3, a ligand that specifically binds with CD83, and any combination thereof.

6. The cord blood derived T cell of claim 1, wherein said antigen binding domain binds antigens associated with tumor endothelium.

7. The cord blood derived T cell of claim 6, wherein said antigen binding domain binds antigens selected from a group comprising of: a) TEM-1; b) TEM-2 c) TEM-3 d) TEM-4 e) TEM-5 f) TEM-6 g) TEM-7 h) TEM-8 i) ROBO-4; j) VEGFR2; k) CD109; l) survivin; and m) CD93

8. The cord blood derived T cell of claim 1, wherein said antigen binding domain binds a tumor antigen.

9. The cord blood derived T cell of claim 8, wherein said tumor antigens are selected from a group comprising of: CLPP, 707-AP, AFP, ART-4, BAGE, MAGE, GAGE, SAGE, b-catenin/m, bcr-abl, CAMEL, CAP-1, CEA, CASP-8, CDK/4, CDC-27, Cyp-B, DAM-8, DAM-10, ELV-M2, ETV6, G250, Gp100, HAGE, HER-2/neu, EPV-E6, LAGE, hTERT, survivin, iCE, MART-1, tyrosinase, MUC-1, MC1-R, TEL/AML, and WT-1.

10. The cord blood derived T cell of claim 1, wherein cord blood derived lymphocytes are isolated by means of a density gradient.

11. The cord blood derived T cell of claim 10, wherein CD4 T cells are isolated from said cord blood derived lymphocytes.

12. The cord blood derived T cell of claim 11, wherein said CD4 T cells are isolated by magnetic activated cell sorting.

13. The cord blood derived T cell of claim 10, wherein CD8 T cells are isolated from said cord blood.

14. The cord blood derived T cell of claim 13, wherein said CD8 T cells are isolated by magnetic activated cell sorting.

15. The cord blood derived T cell of claim 10 wherein said cord blood derived T cells are cultured in the presence of interleukin 2.

16. The cord blood derived T cell of claim 10 wherein said cord blood derived T cells are cultured in the presence of interleukin 7.

17. The cord blood derived T cell of claim 10 wherein said cord blood derived T cells are cultured in the presence of interleukin anti-CD3 and anti-CD28.

18. A mesenchymal stem cell expressing a chimeric antigen receptor (CAR) comprised of:
a) an extracellular antigen binding domain;
b) a transcellular domain; and
c) an intracellular domain.

19. The mesenchymal stem cell of claim 18, wherein said antigen binding domain possesses affinity for tumor antigens are selected from a group comprising of: CLPP, 707-AP, AFP, ART-4, BAGE, MAGE, GAGE, SAGE, b-catenin/m, bcr-abl, CAMEL, CAP-1, CEA, CASP-8, CDK/4, CDC-27, Cyp-B, DAM-8, DAM-10, ELV-M2, ETV6, G250, Gp100, HAGE, HER-2/neu, EPV-E6, LAGE, hTERT, survivin, iCE, MART-1, tyrosinase, MUC-1, MC1-R, TEL/AML, and WT-1.

20. The mesenchymal stem cell of claim 19, wherein said intracellular signaling domain is linked to an activator of molecular pathways endowing MSC-1 phenotype.

21. The mesenchymal stem cell of claim 19, wherein said MSC-1 phenotype is enhanced ability to stimulate a mixed lymphocyte reaction as compared to a naïve MSC.

22. The mesenchymal stem cell of claim 19, wherein said MSC-1 phenotype is enhanced ability to inhibit tumor growth as compared to a naïve MSC.

23. The mesenchymal stem cell of claim 19, wherein said MSC-1 phenotype is enhanced ability to stimulate NK cells as compared to a naïve MSC.

24. The mesenchymal stem cell of claim 19, wherein said MSC-1 phenotype is enhanced ability to stimulate a T cell response as compared to a naïve MSC.

25. The mesenchymal stem cell of claim 24, wherein said T cell response is Th1.

26. The mesenchymal stem cell of claim 23, wherein the NK cells are additionally CD94⁺ and CD117⁺.

27. The mesenchymal stem cell of claim 23, wherein the NK cells are additionally CD161.−.

28. The mesenchymal stem cell of claim 23, wherein the NK cells are additionally NKG2D+.

29. The mesenchymal stem cell of claim 23, wherein the NK cells are additionally NKp46+.

30. The mesenchymal stem cell of claim 23, wherein the NK cells are additionally CD226+.

31. The mesenchymal stem cell of claim 23, wherein the NK cells are additionally CD57+.

32. The mesenchymal stem cell of claim 18, wherein said antigen binding domain binds antigens selected from a group comprising of:
a) TEM-1;
b) TEM-2;
c) TEM-3;
d) TEM-4;
e) TEM-5;
f) TEM-6;
g) TEM-7;
h) TEM-8;
i) ROBO-4;
j) VEGFR2;
k) CD109;
l) survivin; and
m) CD93.

33. The mesenchymal stem cell of claim 20, wherein said activator of molecular pathways endowing MSC-1 phenotype is an intracellular domain of the TLR-4 protein.

34. The mesenchymal stem cell of claim 20, wherein said activator of molecular pathways endowing MSC-1 phenotype is the functional portion of said TLR-4 protein which interacts with MyD88 at a sufficient affinity to trigger said MyD88 signal transduction.

35. The mesenchymal stem cell of claim 20, wherein said activator of molecular pathways endowing MSC-1 phenotype is the functional portion of said TLR-4 protein which interacts with TRAM and MAL at a sufficient affinity to trigger said TLR4 signal transduction.

36. The mesenchymal stem cell of claim 20, wherein said activator of molecular pathways endowing MSC-1 phenotype is the functional portion of said TLR-4 protein which interacts with TRAM and MAL at a sufficient affinity to trigger said TLR4 signal transduction.
